(19) United States
(12) Patent Application Publication
(10) Pub. No.: US 2019/0281878 A1
(43) Pub. Date: Sep. 19, 2019

(54) METHOD AND SYSTEM FOR PROVIDING ACTION RECOMMENDATIONS ASSOCIATED WITH KITCHEN APPLIANCES

(71) Applicant: MIDEA GROUP CO., LTD., Foshan (CN)

(72) Inventors: Tian Tang, Sunnyvale, CA (US); Robin Lu, Foshan (CN); Xin Liu, Foshan (CN); Chen Zhang, Foshan (CN); Dongyan Wang, Foshan (CN)

(21) Appl. No.: 15/925,604

(22) Filed: Mar. 19, 2018

(51) Int. Cl. A23L 33/00; G16H 20/60; G06F 17/30; A61B 5/00

(52) U.S. Cl. CPC A23L 33/30; A61B 5/4866; G06F 17/3097; G16H 20/60

(57) ABSTRACT

A method includes: generating a first nutritional plan for a first user to be implemented in a first time period; monitoring events relevant to real-world implementation of the first nutritional plan by the first user; detecting, via a first kitchen appliance, a first user action associated with the first kitchen appliance; determining a first deviation between the first nutritional plan and a projected nutritional outcome derived based on the monitored events that occurred during a first portion of the first time period up to detection of the first user action; in accordance with the first deviation, revising the first nutritional plan to obtain a second nutritional plan to be implemented in a remainder portion of the first time period; generating a first suggestion regarding a second user action associated with the first kitchen appliance based on the second nutritional plan; and outputting the first suggestion via the first kitchen appliance.

### FIELD OF THE TECHNOLOGY

The present disclosure relates to the field of smart appliances and voice-based digital assistants, and in particular, to providing action recommendations associated with kitchen appliances.

### BACKGROUND OF THE TECHNOLOGY

As smart devices become more and more prevalent, users can utilize various apps installed on their smart devices to record various events of their lives, their goals and wishes, and receive intelligent reminders and suggestions on their smart devices to achieve these goals and wishes. For example, some apps are connected to exercise equipment or wearable devices that monitor a user's activity level throughout the day, and provide reminders and summaries for the user regarding his/her activities. Similarly, some lifestyle apps allow the user to record his/her food intake throughout the day, and his/her moods and health data, to meet certain health goals and/or dietary goals. However, these apps still largely rely on the users to manually enter the goals and constraints that they wish to set, and rely on the users themselves to review how they are doing with respect to the various goals and constraints and make any adjustments in their actions. Because the users have to drive the use of the health and lifestyle apps on their smart devices, except for a small percentage of particularly motivated users, most users may download the apps, but may fail to follow through after a short period of time.

Today, voice-based artificial intelligence (e.g., chatbots or voice-based digital assistants) starts to get integrated into smart devices and some state-of-the-art home appliances. The state-of-the-art voice-based artificial intelligence still focuses on understanding the user's intent in controlling the functions of a smart device and/or a smart home appliance (e.g., through natural language processing of the user's voice commands). The users are still the drivers of the conversations between the devices and the users, and the conversations focus on controlling the functions of the devices in a hands-free manner. Even if the smart devices do provide reminders and alerts based on various triggers (e.g., time, location, schedule, battery life, etc.), the reminders and alerts are usually preset by the users. Thus, truly proactive action recommendations for the users that are provided based on machine intelligence are still lacking, particularly in health and lifestyle management fields.

For these reasons, better action recommendation systems that provide proactive, timely, and appropriate action recommendations to users for improved health and lifestyle purposes are desirable.

### SUMMARY

As set forth in the background section, there is a need for proactive, timely, and appropriate action recommendations for users for improved health and lifestyle purposes. Accordingly, the present disclosure provides methods and system for providing action recommendations associated with kitchen appliances based on various types of information and event triggers. The action recommendations center around actions associated with kitchen appliances, including, for example, refrigerator and pantry restocking recommendations, food ingredient recommendations, recipe recommendations, portion control recommendations, cooking method recommendations, health goal and constraint adjustment recommendations, etc. The reasons that health and lifestyle related action recommendations are particularly pertinent in the kitchen setting is that, usually people make food choices (e.g., grocery restocking, ingredient selection for meals, meal preparation, portion control, etc.) while they are in the kitchen, and they are more likely to follow a food related action recommendation while they are in the kitchen setting. Furthermore, people are usually in a relax mood while they are preparing meals, cleaning up, and/or eating a meal, and they are more likely to be interactive with the voice-based digital assistant, and more likely to provide relevant feedback and information regarding their health and lifestyle preferences, needs, and challenges. Based on these real-time user actions, requirements, and feedback information, better action recommendations can be provided to the user that benefit the user's long term health and lifestyle goals.

In some embodiments, a method, comprising: at a computing device comprising one or more processors and memory: generating a first nutritional plan for a first user to be implemented in a first time period, wherein the first nutritional plan includes a first set of nutritional goals and constraints; monitoring events that are relevant to real-world implementation of the first nutritional plan by the first user; detecting, via a first kitchen appliance, a first user action associated with the first kitchen appliance; determining a first deviation between the first nutritional plan and a projected nutritional outcome that is derived based on the monitored events that occurred during a first portion of the first time period up to detection of the first user action; in accordance with the first deviation, revising the first nutritional plan to obtain a second nutritional plan to be implemented in a remainder portion of the first time period, wherein the second nutritional plan includes a second set of nutritional goals and constraints that are distinct from the first set of nutritional goals and constraints; generating a first suggestion regarding a second user action associated with the first kitchen appliance based on the second nutritional plan; and outputting the first suggestion via the first kitchen appliance.

In accordance with some embodiments, a system includes one or more processors, and memory storing one or more programs; the one or more programs are configured to be executed by the one or more processors and the one or more programs include instructions for performing or causing performance of the operations of any of the methods described herein. In accordance with some embodiments, a non-transitory computer readable storage medium has stored therein instructions, which, when executed by an electronic device, cause the device to perform or cause performance of the operations of any of the methods described herein.

Various advantages of the presently disclosed technology are apparent in light of the descriptions below

### BRIEF DESCRIPTION OF THE DRAWINGS

The aforementioned features and advantages of the disclosed technology as well as additional features and advantages thereof will be more clearly understood hereinafter as a result of a detailed description of preferred embodiments when taken in conjunction with the drawings.

To describe the technical solutions in the embodiments of the present disclosed technology or in the prior art more clearly, the following briefly introduces the accompanying drawings required for describing the embodiments or the prior art. Apparently, the accompanying drawings in the following description show merely some embodiments of the present disclosed technology, and persons of ordinary skill in the art may still derive other drawings from these accompanying drawings without creative efforts.

FIG. 1 is a block diagram illustrating an operating environment for a digital assistant that provides action recommendations associated with kitchen appliances in accordance with some embodiments.

FIG. 2 is a block diagram illustrating a digital assistant server that provides action recommendations associated with kitchen appliances in accordance with some embodiments.

FIG. 3 illustrates a usage scenario of providing action recommendations in a smart kitchen environment in accordance with some embodiments.

FIG. 4 illustrates an example of processing various types of information to refine action recommendations over time in accordance with some embodiments.

FIG. 5 is a flowchart diagram of a method for providing action recommendations associated with kitchen appliances in accordance with some embodiments.

FIG. 6 is a block diagram of a server system for providing action recommendations associated with kitchen appliances in accordance with some embodiments.

Like reference numerals refer to corresponding parts throughout the several views of the drawings.

### DESCRIPTION OF EMBODIMENTS

Reference will now be made in detail to embodiments, examples of which are illustrated in the accompanying drawings. In the following detailed description, numerous specific details are set forth in order to provide a thorough understanding of the subject matter presented herein. But it will be apparent to one skilled in the art that the subject matter may be practiced without these specific details. In other instances, well-known methods, procedures, components, and circuits have not been described in detail so as not to unnecessarily obscure aspects of the embodiments.

The following clearly and completely describes the technical solutions in the embodiments of the present application with reference to the accompanying drawings in the embodiments of the present application. Apparently, the described embodiments are merely a part rather than all of the embodiments of the present application. All other embodiments obtained by persons of ordinary skill in the art based on the embodiments of the present application without creative efforts shall fall within the protection scope of the present application.

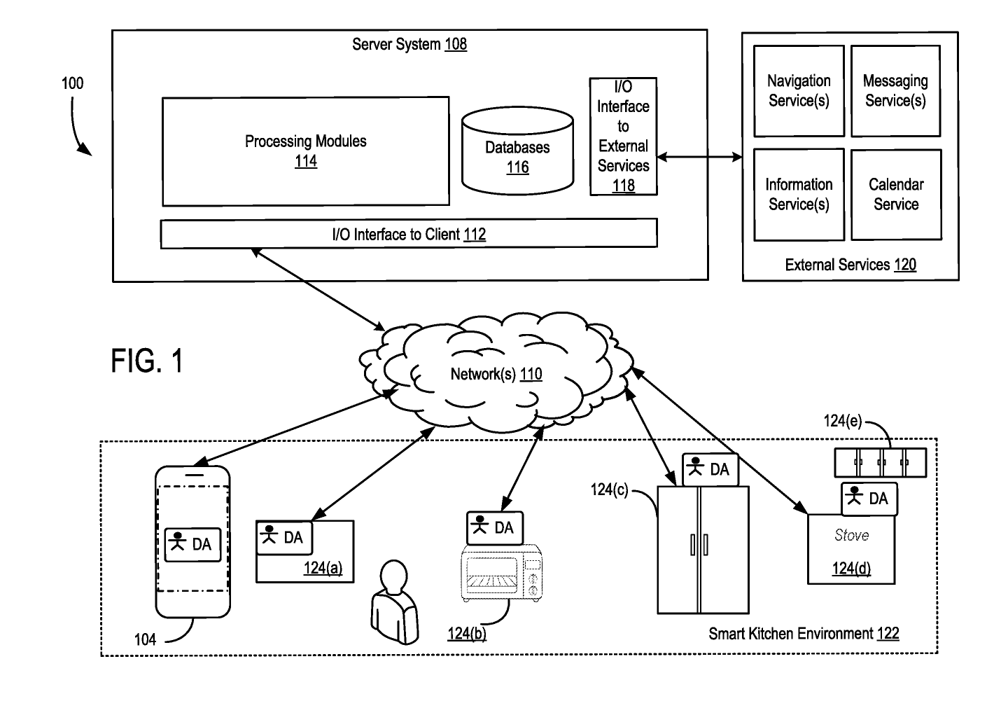

FIG. 1 is a block diagram of an operating environment 100 for a voice-based digital assistant that provides action recommendations associated with kitchen appliances in accordance with some embodiments.

The operating environment 100 is implemented according to a client-server model. The operating environment 100 includes a smart kitchen environment 122 and a server system 108 communicatively coupled with the smart kitchen environment 122 via cloud networks 110. In some embodiments, the smart kitchen environment 122 includes one or more smart kitchen appliances 124. Examples of the smart kitchen appliances 124 include refrigerators 124(c), freezers, microwave ovens 124(b), stoves 124(d), toaster ovens, convection ovens 124(a), electric cookers, grill pans, smart storage cabinets 124(e), etc. In some embodiments, the client-side environment 100 further includes a user device 104 (e.g., a smartphone, a tablet, a personal computer, or a central communication hub).

In some embodiments, a respective kitchen appliance of the one or more kitchen appliances 124 includes an input/output user interface. The input/output user interface optionally includes one or more output devices that enable presentation of media content, including one or more speakers and/or one or more visual displays. The input/output user interface also optionally includes one or more input devices, including user interface components that facilitate user input, such as a keypad, a voice-command input unit or microphone, a touch screen display, a touch-sensitive input pad, a gesture capturing camera, or other input buttons or controls.

In some embodiments, a respective kitchen appliance further includes sensors, which senses operating environment information of the respective kitchen appliance. Sensors include but are not limited to one or more light sensors, cameras (also referred to as image sensors), humidity sensors, temperature sensors, motion sensors, weight sensors, spectrometers, and other sensors. In some embodiments, the sensors associated with various kitchen appliances are used to provide user presence information (e.g., location of the user in the kitchen, and which appliance(s) that the user is currently interacting with, etc.), food item inventory information, food preparation status information, etc. In some embodiments, the sensors also provide information on the indoor environment, such as temperature, and humidity of the rooms in the house.

In some embodiments, one or more devices and/or appliances in the client-side kitchen area includes a respective digital assistant (DA) client, and a DA server is executed on the server system 108. Each device 124 that includes an audio input interface (e.g., a microphone) can serve as a voice input endpoint device for the digital assistant, and captures voice inputs uttered by a user. The user can move about the smart kitchen environment 122, and multiple devices 124 that are located in the vicinity of the user can capture the same voice input, and independently transmit the voice input to the server system 108 through their own communication channels to the digital assistant server. The DA client provides client-side functionalities such as user-facing input and output processing and communications with the DA server 106. The DA server 106 provides server-side functionalities for any number of DA clients each residing on a respective user device 104 (e.g., user devices registered for a household account) and/or a respective kitchen appliance 124. The user can speak directly to the DA client on a respective user device or kitchen appliance to interact with the digital assistant on the server system 108.

In some embodiments, the server system 108 includes one or more processing modules 114, data and models 116, an I/O interface to client 112, and an I/O interface to external services 118. The client-facing I/O interface 112 facilitates the client-facing input and output processing for the server system 108. For example, when the same voice input is transmitted to the server system 108 via multiple independent communication channels from multiple kitchen appliances with voice input interfaces, the client-facing I/O interface 112 selects the input stream received from one of the multiple communication channels based on input quality, or integrate the input streams from the multiple communication channels to obtained a better quality input stream. Then, the client-facing I/O interface 112 provides the best quality voice input that has been obtained to the processing modules 114 for natural language processing and intent deduction. Similarly, when the digital assistant server 108 provides an action recommendation to the user or sends a related machine command to a kitchen appliance or external service (e.g., recommendation or machine command for changing cooking temperatures, cooking styles, or executing a restocking order, etc.), the digital assistant also chooses the output channel that is most appropriate under the circumstance (e.g., based on the user's current action (e.g., cooking vs. eating) and the user's location in the kitchen (e.g., in front of the stove vs. near the refrigerator, etc.)).

The database and models 116 include various user data for each user and/or household of users, such as individual user's account data, health data, diet preferences and restrictions, activity data, health goals, lifestyle, schedule constraints, etc. The one or more processing modules 114 utilize the data and models 116 to monitor events that are relevant to the user's health and lifestyle goals and constraints, to determine a compliance level of certain nutritional plan based on past events, to generate and update the nutritional plan and recommendation strategies regarding user actions associated with the one or more kitchen appliances.

In some embodiments, the server system 108 also communicates with external services 120 (e.g., navigation service(s), messaging service(s), information service(s), calendar services, home appliance control service(s), social networking service(s), recipe services, nutritional information services, shopping services, etc.) through the network(s) 110 for task completion or information acquisition. The I/O interface to the external services 118 facilitates such communications.

In some embodiments, the server system 108 can be implemented on at least one data processing apparatus and/or a distributed network of computers. In some embodiments, the server system 108 also employs various virtual devices and/or services of third party service providers (e.g., third-party cloud service providers) to provide the underlying computing resources and/or infrastructure resources of the server system 108.

Examples of the communication network(s) 110 include local area networks (LAN) and wide area networks (WAN), e.g., the Internet. The communication network(s) 110 may be implemented using any known network protocol, including various wired or wireless protocols, such as Ethernet, Universal Serial Bus (USB), FIREWIRE, Global System for Mobile Communications (GSM), Enhanced Data GSM Environment (EDGE), code division multiple access (CDMA), time division multiple access (TDMA), Bluetooth, Wi-Fi, voice over Internet Protocol (VoIP), Wi-MAX, or any other suitable communication protocol.

In some embodiments, there is a network router that connects the different devices and appliances in the smart kitchen environment 122 to the network 110, and routes the communications in and out of the smart kitchen environment to and from the digital assistant server. The network router does not intelligently process the communications that goes in and out of the smart kitchen environment for purposes other than data communication (e.g., routing the messages to their destinations based on the addresses specified in the communications) and the network router is considered to be part of the network 110, as opposed to part of the controlled devices or the digital assistant client or server.

In some embodiments, the smart kitchen environment 122 has a centralized cloud account for each household which manages all registered kitchen appliances 124 that are associated with the smart kitchen environment of that household and that are reachable/controllable over the network 110. The kitchen appliances 124 are required to comply with an API to communicate with the server system 108. Once the server system 108 receives an input (e.g., a triggering event such as a user action associated with a kitchen appliance or a user voice input), no matter which particular kitchen appliance transmitted the input to the server system, the server system will analyze the input and determine the intent of the input and send a command and/or audio feedback to a kitchen appliance, wherein the kitchen appliance playbacks the audio output or performs a requested task in accordance with the command. As long as the input is captured by one of the kitchen appliances 124 that has an input interface and that is network enabled (e.g., the user does not need to be near any particular device that it needs to control, or be near a central communication hub), the digital assistant (DA) can be activated and control any of the kitchen appliance 124 based on the input.

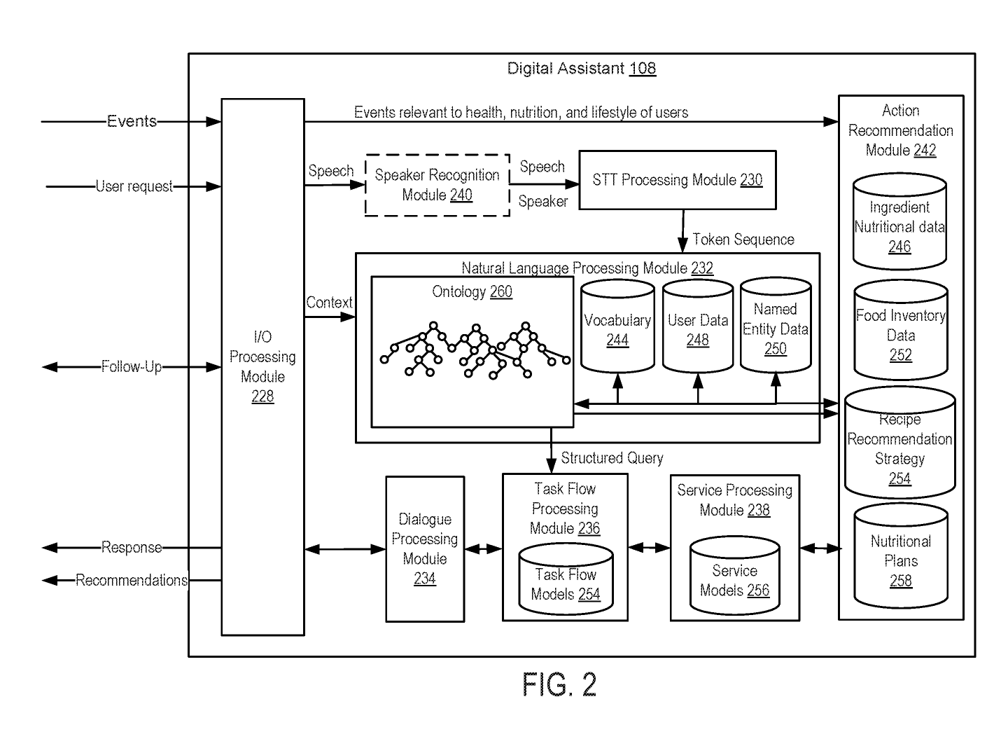

FIG. 2 is a block diagram of an exemplary digital assistant server system 108 in accordance with some embodiments. It should be noted that the digital assistant system 108 is only one example of a digital assistant system, and that the digital assistant system 108 may have more or fewer components than shown, may combine two or more components, or may have a different configuration or arrangement of the components. The various components shown in FIG. 2 may be implemented in hardware, software, firmware, including one or more signal processing and/or application specific integrated circuits, or a combination of thereof. The digital assistant system 108 includes memory, one or more processors, one or more input/output (I/O) interfaces, and a network communications interface. These components communicate with one another over one or more communication buses or signal lines.

As shown in FIG. 2, the digital assistant server 108 includes an I/O processing module 228, a speaker recognition module 240, a speech-to-text processing module 230, a natural language processing module 232, a task flow processing module 236, a dialogue processing module 234, a service processing module 238, and an action recommendation module 242 (e.g., all part of processing modules 114 in FIG. 1). These processing modules utilize, directly or indirectly, the various databases and models 116, including, for example, vocabulary 244, user data 248, named entity data 250, ingredient nutritional data 246, food inventory data 252, recipe/portion/ingredient recommendation strategies 254, and nutritional plans 258, etc. to understand the user's intent, to obtain useful information regarding user's health and lifestyle needs, preferences, and goals, to provide suitable recommendations and needed information, and to perform requested tasks.

As shown in FIG. 2, in some embodiments, the I/O processing module 228 interacts with the user through a user device (e.g., a user device 104 in FIG. 1) and/or other devices (e.g., kitchen appliances 124 in FIG. 1). The I/O processing module 228 utilizes the network communications interface to obtain user input (e.g., a speech input), to provide responses to the user, to obtain event information that are relevant to the user's health and lifestyle and implementation of the user's nutritional plans, and to provide action recommendations associated with the kitchen appliances 124. In some embodiments, the I/O processing module 228 also interacts with the user (e.g., follow-up) to elicit information and request clarifications through multiple speech-based exchanges.

In some embodiments, the I/O processing module 228 optionally obtains information associated with trigger events and/or user inputs, along with or shortly after the detection of the trigger events and/or receipt of the user inputs. The information includes user-specific data, activity data, health data, feedback data from other members of household, user-specific vocabulary, and/or user preferences relevant to the user input and user's health and lifestyle. The trigger events include, for example, detection of the user in the kitchen or in front of a kitchen appliance, the user opening or operating a kitchen appliance, and/or the user interacting with a kitchen appliance (e.g., cleaning the kitchen appliance). The trigger events may also include particular software and hardware states of the devices 124, such as the food inventory states (e.g., inventory low, or nutritionally unbalanced), food consumption data, appliance usage states, door open/closed states, appliance on/off states, etc. In some embodiments, the trigger event information is received at the time the user request is received. Information related to the surrounding environment of the user at the time of the user request may also be received. In some embodiments, speech inputs may be received from the user in conjunction with or independent of the event information and/or context information.

In some embodiments, the I/O processing module 228 sends follow-up questions to, and receives answers from, the user regarding the user request, user's preferences, user's health and lifestyle, and user's activities, etc. In some embodiments, when a user request or feedback is received by the I/O processing module 228 and the user request or feedback contains a speech input, the I/O processing module 228 forwards the speech input to the speaker recognition module 240 for speaker recognition and subsequently to the speech-to-text (STT) processing module 230 for speech-to-text conversion.

In some embodiments, person-specific speech-to-text models are selected to perform the speech-to-text conversion based on the speaker recognition result. In some embodiments, people's identity information and relationship in a household are stored in the user data database 248. Based on the identity information and relationship information, the digital assistant 108 can properly associated feedback received from one household member regarding health, activities, preferences, and goals for another household member with that other household member, and use the information to provide action recommendations relevant to that other household member. In some embodiments, the named entity data 250 is used to identify people when nicknames are used to refer oneself or other members of the household.

In some embodiments, the speech-to-text processing module 230 receives speech input (e.g., a user utterance captured in a voice recording) through the I/O processing module 228 or the speaker recognition module 240. In some embodiments, the speech-to-text processing module 230 uses various acoustic and language models to recognize the speech input as a sequence of phonemes, and ultimately, a sequence of words or tokens written in one or more languages. The speech-to-text processing module 230 is implemented using any suitable speech recognition techniques, acoustic models, and language models, such as Hidden Markov Models, Dynamic Time Warping (DTW)-based speech recognition, and other statistical and/or analytical techniques. In some embodiments, the speech-to-text processing can be performed at least partially by a third party service or on the user's device. Once the speech-to-text processing module 230 obtains the result of the speech-to-text processing (e.g., a sequence of words or tokens), it passes the result to the natural language processing module 232 for intent deduction or information item extraction.

In some embodiments, the natural language processing module 232 of the digital assistant 108 takes the sequence of words or tokens (“token sequence”) generated by the speech-to-text processing module 230, and attempts to associate the token sequence with one or more “actionable intents” recognized by the digital assistant, or one or more information items relevant to a user's health and lifestyle needs, preferences, and actions. As used herein, an “actionable intent” represents a task that can be performed by the digital assistant 108 and/or devices subject to control by the digital assistant, and that has an associated task flow implemented in the task flow models 354. The associated task flow is a series of programmed actions and steps that the digital assistant system takes in order to perform the task. The information items that are relevant to the user's health and lifestyle needs, preferences, and actions are provided to the action recommendation module 242. The information item includes, for example, the user's preference for a meal, the user's acceptance or refusal of a recommendation, the user's nutritional goals (e.g., calories, nutritional composition of meals, etc.), the user's health data (e.g., heart rates, general well-being, moods, etc.), the user's activities and schedules (e.g., worked out, tired, only having 20 minutes for lunch, etc.).

In some embodiments, the information items and the user's intent that are derived from the natural language processing are sent to the action recommendation module 242, where the action recommendation module uses the information items and user intent, as well as the event information, and information and strategies stored in the ingredient nutritional database 246, the food inventory database 252, the recipe recommendation strategy 254, and the nutritional plans database 258, to generate the action recommendations for the user. In some embodiments, the action recommendation module 242 provides action recommendations on which types and what quantity of food ingredients to use for meal preparation, step-by-step instructions for cooking a meal, portion and ingredient form recommendations (e.g., sliced, diced, pureed, etc.) for cooking a meal, food consumption order recommendation (e.g., fruit and veggies before starch, soup before entree, etc.), exercise recommendations (e.g., “If you eat food A for desert, you need to walk 20 minutes afterwards; and if you eat food B for desert, you need to walk for one hour afterwards.”), food restocking recommendations, ingredient substitution recommendations, recipe substitution recommendations, nutritional plan revision recommendations, and any of the above recommendations regarding the whole household or another member of the household, etc.

In some embodiments, the natural language processing module 232 optionally uses the context information that has been received to clarify, supplement, and/or further define the deduced intent, and generates a structured query that is provided to the task flow processing module 236 which uses the structured query to select and execute the relevant task flows in the task flow models 254. In some embodiments, the task flow models that are chosen includes task flow models for controlling an appliance (e.g., turning on and off, turning up the temperature, adjusting cooking time, etc.), reading recipes and instructions via a speech output interface, providing information requested, and/or executing online shopping, restocking food, etc.

In some embodiments, the natural language processing is based on an ontology 260. The ontology 260 is a hierarchical structure containing a plurality of nodes, each node representing either an “actionable intent”, “information item”, or a “property” relevant to one or more of the “actionable intents”, “information item”, or other “properties.” As noted above, an “actionable intent” represents a task that the digital assistant system is capable of performing (e.g., a task that is “actionable” or can be acted on). A “property” represents a parameter associated with an actionable intent or a sub-aspect of another property. An “information item” includes a defined parameter in one of the databases that are used by the action recommendation module 242 to provide action recommendations. A linkage between an actionable intent node and a property node in the ontology 360 defines how a parameter represented by the property node pertains to the task represented by the actionable intent node. “An information item” can be an intent or a property.

In some embodiments, the natural language processing module 232 shown in FIG. 2 receives the token sequence (e.g., a text string) from the speech-to-text processing module 330, and determines what nodes are implicated by the words in the token sequence. In some embodiments, if a word or phrase in the token sequence is found to be associated with one or more nodes in the ontology 260 (via the vocabulary index 244), the word or phrase will “trigger” or “activate” those nodes. When multiple nodes are “triggered,” based on the quantity and/or relative importance of the activated nodes, the natural language processing module 232 will select one of the actionable intents as the task (or task type) that the user intended the digital assistant to perform or as one of the information items to pass to the action recommendation module 242.

In some embodiments, the digital assistant system 108 stores names of specific entities in the named entity database 250, so that when one of these names is detected in the user request and/or user feedback, the natural language processing module 232 will be able to recognize that the name refers to a specific instance of an information item, a property or sub-property in the ontology. In some embodiments, the names of specific entities are names of businesses, restaurants, people, movies, food ingredients, recipe names, cooking method names, and the like. In some embodiments, the named entity database 250 also includes the aliases of the home appliance devices that are provided by individual users during the device registration stage for the different home devices. In some embodiments, the digital assistant system can search and identify specific entity names from other data sources, such as the user's address book or contact list, a musicians database, a recipe database, a grocery store database, and/or a restaurant database, etc. In some embodiments, a trigger phrase for waking up the digital assistant is stored as a named entity, so that it can be identified and given special significance when it exists in the speech input of the user.

User data 348 includes user-specific information, such as user-specific vocabulary, user's demographic data, user preferences, user's addresses, user's default and secondary languages, user's contact list, and other short-term or long-term information for each user. The natural language processing module 232 can use the user-specific information to supplement the information contained in the user input to further define the user intent. In some embodiments, the user data also includes the user's specific voiceprint for user authentication or speech samples for speaker recognition.

In some embodiments, when the natural language processing module 232 passes the structured query (including any completed parameters) to the task flow processing module 236 (“task flow processor”), the task flow processing module 236 performs the actions required to “complete” the user's ultimate request. In some embodiments, the various procedures necessary to complete these tasks are provided in task flow models 254. In some embodiments, the task flow models 254 include procedures for obtaining additional information from the user, and task flows for performing actions associated with the actionable intent. In some embodiments, the task flows in the task flow models 254 describe the steps for controlling each home appliance device that is registered with the home assistant, and based on the device name list, the digital assistant operates to execute the steps in a suitable task flow with respect to the home appliance device that is specified by the alias of the home device in the user's speech input. In some embodiments, the steps to perform a requested task includes a list of encoded instructions that are to be sent to a controlled device (e.g., a home appliance) over the network, such that the controlled device executes those encoded instructions to accomplish the required intent of the user.

In some embodiments, the task flow processor 336 employs the assistance of a service processing module 338 (“service processor”) to complete a task requested in the user input or to provide an informational answer requested in the user input. For example, the service processing module 338 can act on behalf of the task flow processing module 336 to send a command to a home appliance, make a phone call, set a calendar entry, invoke a map search, invoke or interact with other user applications installed on the user device, and invoke or interact with third party services (e.g. a restaurant reservation portal, a social networking website or service, a banking portal, online shopping portal, etc.,). In some embodiments, the protocols and application programming interfaces (API) required by each service can be specified by a respective service model among the service models 356. The service processor 338 accesses the appropriate service model for a service and generates requests for the service in accordance with the protocols and APIs required by the service according to the service model.

In some embodiments, the action recommendation module 242 provides the instructions to the appliances in the user's smart home for monitoring the user's speech input and events that are relevant to the user's health, lifestyle, and implementation of the user's nutritional plan. The action recommendation module 242 optionally provides the instructions to initiate and carry out conversations with the users in the household regarding the users' respective activities, health, lifestyle, and implementation of the users' respective nutritional plans, and/or regarding the same for one or more other members of the users' households. The action recommendation module 242 further provides the instructions to the appliances to monitor events that indicate suitable opportunities to initiate and carry out the above conversations, such as when the user is preparing a meal in the kitchen (e.g., as opposed to getting a beer and then go to the living room to watch TV or entertain guests), or when the user is eating alone (e.g., as opposed to eating with family or guests). In some embodiments, the action recommendation module 242 further includes instructions to review and modify the user's nutritional plan after a period of monitoring the user's actual implementation of the nutritional plan, such that the goals and limitations set forth in the nutritional plan are more suitable to the user's current situation and are more realistic to the user's taste preferences and lifestyle. In some embodiments, the action recommendation module 242 further includes instructions for determining suitable food ingredients, cooking styles, recipes, and/or portion sizes, etc. for meal preparation to implement the user's nutritional plan based on the food inventory at the user's house, the user's food preferences, the user's recent dietary compositions, the user's recent activities, the user's recent health and mood data, and/or the user's schedules, etc. In some embodiments, the action recommendation module 242 includes instructions for providing substitution recommendations (e.g., providing ingredient substitution recommendations, cooking method substitution recommendations, recipe substitution recommendations, and/or food restocking or shopping substitution recommendations, etc.) based on the user's current actions and preferences, the user's current food choices and actions, the food inventory at the user's house, the user's food preferences, the user's recent dietary compositions, the user's recent activities, the user's recent health and mood data, and/or the user's schedules, etc. In some embodiments, the action recommendation module 242 optionally performs actions proactively, such as restocking the refrigerator with healthy ingredients that the user is more likely to accept than forcing the user to purchase those items himself/herself.

In some embodiments, the natural language processing module 232, dialogue processing module 234, task flow processing module 236, and action recommendation module 242 are used collectively and iteratively to deduce and define the user's intent, to obtain information to further clarify and refine the user intent and goals, to obtain user's preference, approval, constraints, health data, and/or schedule data, etc., and to finally generate a response, execute an action, and/or provide a recommendation, to fulfill the user's intent and help the user achieve his/her health and lifestyle goals.

In some embodiments, the additional dialogue with the user in order to obtain additional information, disambiguate potentially ambiguous utterances, and provide action recommendations, is performed by the dialogue processing module 234. In some embodiments, the dialogue processing module 234 determines how (and/or when) to ask the user for the additional information, and receives and processes the user responses.

In some embodiments, the questions are provided to and answers are received from the users through the I/O processing module 228. In some embodiments, the dialogue processing module 234 generates the audio outputs embodying the questions and/or recommendations, and sends the audio outputs to an output endpoint device selected by the digital assistant where the selected output device presents the questions and/or recommendations to the user. The user's responses are captured by one or more input endpoint devices and transmitted to the digital assistant, where the digital assistant processes the voice responses received from a selected input endpoint device and obtains the required clarifications from the voice responses. During the multiple exchanges between the digital assistant and the user, the user could be moving around in the house and listening to the digital assistant's outputs from different output endpoint devices 124, and having his/her answers picked up by different sets of one or more input endpoint devices 124, depending on the locations of the user when those outputs are delivered to the user and when the user provided his/her answers to the digital assistant. Once answers are received from the user, the dialogue processing module 334 populates the structured query with the missing information, or passes the information to the action recommendation module 242 to complete the action recommendation process.

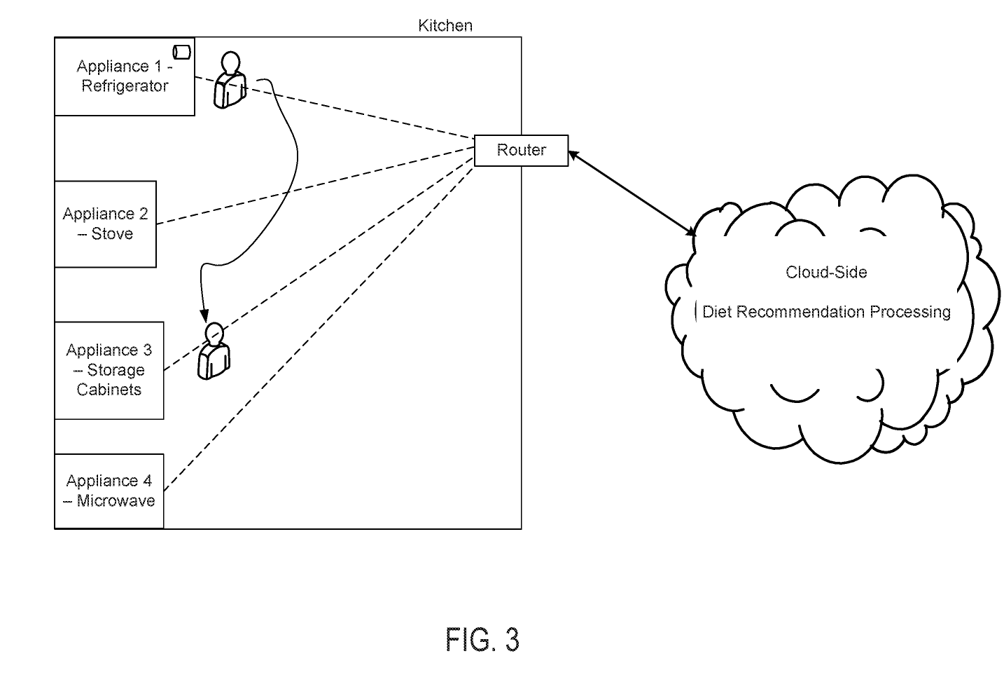

FIG. 3 illustrates a usage scenario of providing action recommendations (e.g., diet recommendations) in the smart kitchen environment in accordance with some embodiments. In some embodiments, the server system 108 has generated a nutritional plan based on the user's goals (e.g., losing weight, building muscles, getting more energy, becoming more limber, reducing cholesterol, reducing blood sugar, etc.) and preferences (e.g., slowly and steadily with minimal lifestyle changes, fast and quick results, using diet-focused method vs activity-focused method, low impact on poor joints, no salads, keeping deserts, etc.), and the user's health data (e.g., age, sex, weight, height, body fat index, heart condition, cholesterol, blood sugar, long-term disabilities, short-term disabilities, chronic diseases, current illnesses, etc.). The nutritional plan is established for a predefined period of time (e.g., one month, or one year) with corresponding goals (e.g., losing 20 pounds, reducing blood sugar level by 10, reducing blood pressure to normal range, decreasing BMI by x amount, etc.) and restrictions (e.g., losing no more than 5 pounds per week, maintaining a balanced diet (e.g., no low-carb meals), no extraneous exercises, etc.). The goals of the nutritional plan may include a number of calories that the user can consume per day, the balance of nutrients (e.g., the ratio between fat, carbs, protein, and various vitamins and minerals, etc.) in the user's diet, and/or the relative proportion of calorie intake (e.g., from meals, snacks, and drinks) and calorie expenses (e.g., baseline body functions, exercises, daily activities, etc.).

In some embodiments, the server system 108 constantly monitors user data and events that are relevant to the implementation of the nutritional plan by the user. For example, the server system 108 collects user's updated health data (e.g., weight, blood pressure, blood sugar, body mass index, exercise records, etc.) from the user device 104. The server system 108 may also collect user's diet data from one or more kitchen appliances. For example, based on what have been taken from the fridge and what have been cooked on the stove or microwave oven, the server system 108 can obtain calories information and/or nutrients information based on the detected ingredients. The server system 108 may further detect inventory data, such as what food ingredients are stored in the fridge and/or the food storage cabinets. In some embodiments, the server system 108 monitors and records the user's usual meal time based on the user's locations, and time spent at restaurants and in the kitchen. In some embodiments, the server system 108 monitors and records the user's cooking methods based on the states and settings used on the cooking appliances in the kitchen.

The server system 108 evaluates a performance of the formerly formed nutritional plan (e.g., the effectiveness of the plan, how well the user likes the plan, how well the user has implemented the plan, etc.) based on the monitored data and user feedback from earlier portion(s) of the time period for the plan. In some embodiments, the server system 108 generates a summary to evaluate how well the user has been following the nutritional plan during the past two days or one week. For example, the server system 108 compares the calories of food that have been consumed by the user and the targeted calories for those meals provided in the nutritional plan, and evaluates whether the user has been eating heavier meals than he/she is supposed to. The server system 108 also monitors whether the user has repeatedly rejected meal recommendations generated based on the current nutritional plan, and whether the user or his/her family members have provided any reasons for the user's rejection of those recommendations. In some embodiments, if the server system detects that based on the current implementation trend, that the outcome of the implementation will deviate from the nutritional plan significantly, or if the user has expressed that there is certain difficulty in implementing the current nutritional plan based on current circumstances (e.g., time constrains, lack of motivation, presence of house guests, health problems, etc.), the server system modifies at least one aspects of the nutritional plan, such as the calorie requirements, health goals, nutritional balance and/or constraints, and the strategies (e.g., diet-focused vs. exercise-focused strategies) for implementing nutritional plan. The server system 108 then updates the nutritional plan based on the deviation and reasons for deviation. The server system 108 saves the updated nutritional plan as a second nutritional plan to be implemented in the subsequent portions of the time period. The reason that the server system should revise the nutritional plan based on intermediate evaluations of the performance and actual implementations of the original nutritional plan is that, continuing the push for the same nutritional plan and making it even harder to implement after the user has failed to implement it successfully to begin with (e.g., by imposing further calorie restrictions in order to meet earlier goals after the user has already exceeded the earlier calorie quotas), the user is more likely to give up all together and render the original plan meaningless. By revising the nutritional plan for the remainder of the time period such that it is more realistic for the user to accomplish, it would be more beneficial to the user's health and lifestyle goals (e.g., smaller improvement is better than complete failure). In addition, by interacting with the user via dialogues and monitoring the points of failure in the user's actual implementation of the current nutritional plan (e.g., poor ingredient choice, poor cooking method choices, poor portion choices, irregular meal time, etc.), the digital assistant server is able to revise the nutritional plan in a manner that will make the implementation of the revised plan more realistic and sometimes more effective, which ultimately benefits the user and gets the user closer to his/her health goals.

As shown in the example scenario in FIG. 3, a presence sensor (e.g., an image sensor, a light sensor, and/or a motion sensor) located on the refrigerator door or inside the refrigerator compartment detects a user's presence and action in front of the refrigerator (e.g., that the user just opened the refrigerator and took out some ingredients). The sensors inside the refrigerator (e.g., image sensors, and weight sensors) determine which ingredients and what quantities of ingredients the user has taken out of the refrigerator. Based on the ingredients, the current time, and the current nutritional plan (e.g., this could be the unrevised plan if the criteria for revising the nutritional plan has not been met, or the revised plan if the criteria for revising the plan has been met), the digital assistant generates one or more action recommendations associated with the refrigerator. The action recommendations optionally include what ingredients to take out of the refrigerator (e.g., “For lunch preparation, please take out one head of lettuce, one tomato, one block of tofu.”). In some embodiments, the digital assistant takes into account of what the user has already taken out of the refrigerator, and provides a recommendation that compliments the user's existing ingredients and still meets the nutritional requirements of the current nutritional plan (e.g., “It appears that you have taken some sliced cheese out of the refrigerator, you could add some grilled chicken and white bread to make a sandwich.”). In some embodiments, the digital assistant also makes substitution recommendations based on the current nutritional plan and/or the ingredients that the user has taken out (“I see that you have taken some sliced cheese and some grilled chicken out. Your diary intake for the week is a little high. I recommend that you make a sandwich without the cheese.”). The recommendations provided by the digital assistant are optionally output by a voice output interface on the refrigerator or another appliance in the vicinity of the user. In some embodiments, while the user is in front of the refrigerator, the digital assistant may also make recommendations based on the inventory in the refrigerator and composition of the user's recent meals (e.g., “There are some fresh vegetables in the fridge, you may want to compliment your sandwich with a small salad.”). In some embodiments, the digital assistant may also provide a restocking recommendation based on the inventory in the refrigerator and the user's recent meal choices (e.g., “You have been eating a lot of ice creams lately, would you like to buy some similar frozen treats that are lower in calories?). In some embodiments, the digital assistant may also provide recommendations regarding the quantity of ingredients to be taken out of the refrigerator for meal preparation (e.g., “It appears that you have taken three tomatoes out of the fridge. Three tomatoes can be paired with two eggs to create a nutritionally balanced entree.”).

After the digital assistant has provided one or more action recommendations in association with the refrigerator, the digital assistant detects user actions and/or verbal feedback that are indicative of the user's actual actions and opinions of the recommendations. For example, the user may ignore the recommendations regarding ingredients, and take out items that are not among those recommended. The user may ignore the quantity recommendation, and takes out more than the quantity recommended. The user may reject a recommendation due to various reasons (e.g., “No, I don't like quesadillas.” “No, I like full-fat ice creams.” “No, I have no time to cook a hot meal right now.” or “No, mom wants to keep the vegetables for our guests tonight.”). The digital assistant optionally revises the recommendations and provides alternative recommendations based on the user's actions and feedback. The digital assistant will take records the user's actions and feedback and take them into account in subsequent recommendations provided with respect to other actions associated with other kitchen appliances.

Continuing with the above example, after the user has taken out some ingredients from the refrigerator, the digital assistant server detects via the smart stove that the user is now standing in front of the stove with some food ingredients. In response to detecting the user's presence and/or actions (i.e., turning on the stove), the server system 108 provides a recommendation regarding cooking method. For example, the digital assistant server optionally provides step by step cooking instructions based on a recommended recipe (e.g., “Heat 2 ounces of oil to medium high temperature . . . Stir in the grilled chick strips . . . ”). Or the digital assistant provides a recipe substitution recommendation (e.g., “Based on the ingredient you took out, you can make a grilled sandwich with added butter, or you can make an open faced sandwich with toasted bread to reduce the calorie and fat intake.”). Or the digital assistant can provide a cooking method substitution (e.g., “Lower the temperature and reduce cooking time to preserve more nutrients and make the food more filling.” or “These ingredients can be steamed or grilled with minimal cooking oil.”).

In this example, while the user is cooking on the stove, the digital assistant detects that the user has moved to the smart pantry, and an image sensor or a motion sensor located at the storage cabinets of the smart pantry detects that the user has opened the cabinet door for beverages. Based on the cooking methods and ingredients, the digital assistant makes a recommendation for beverages that takes into account of both how the beverage may compliment the cooking method and ingredients, but also how they fit the user's preferences and current nutritional plan. For example, if the user went against the digital assistant's recommendation for cooking methods, and is making a grilled sandwich with fried chicken strips, the digital assistant optionally makes a recommendation for a diet beverage as opposed to regular sugar beverages.

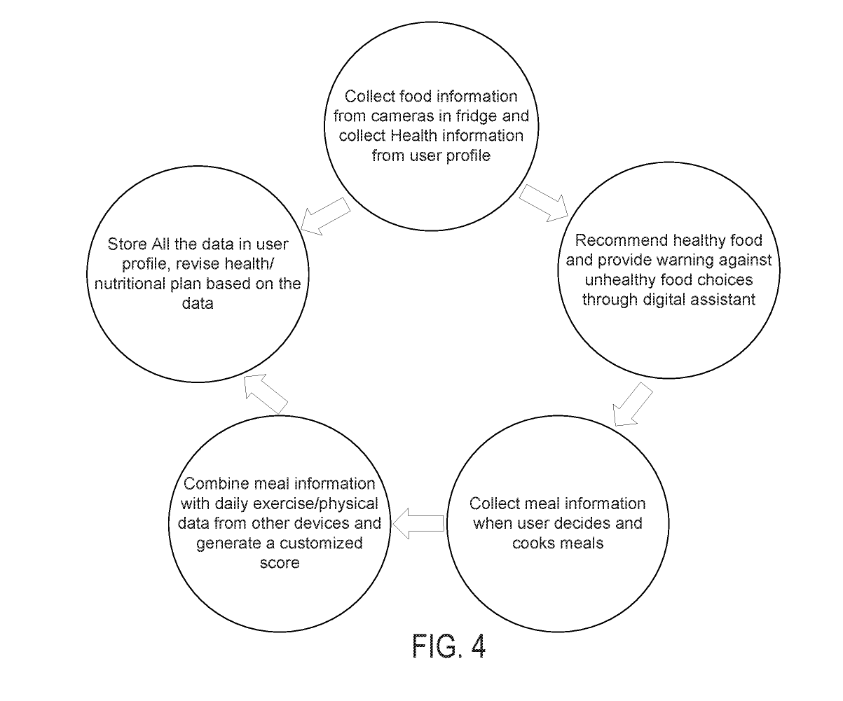

FIG. 4 is a diagram illustrating the iterative process of: (1) gathering information relevant to the implementation of the current nutritional plan, such as collecting food information from cameras in the fridge and collecting user's health information from user profile; (2) making action recommendations at suitable times based on user's actions to implement the current nutritional plan, such as providing ingredient recommendations (e.g., recommending healthy food and warning against unhealthy food choices) via the digital assistant when user is retrieving items from the refrigerator, or making cooking method recommendations (e.g., providing healthy alternative cooking methods to substitute an unhealthy one that the user is going to use) when the user is preparing the meal in front of a stove or microwave; (3) Gather feedback from the user's actions (e.g., what ingredients and cooking methods were actually used vs. those recommended) and verbal inputs, the actual meals prepared and consumed, and determine in what aspects they deviate from the recommendations, and in what aspects they impact the subsequent implementation of the nutritional plan; (4) Projecting an outcome corresponding to the current nutritional plan based on currently gathered data, including meal information, daily exercise data and health data collected from other devices and/or user feedback, and determining a deviation between the projected nutritional outcome and current nutritional plan (e.g., deriving a performance score for the current nutritional plan, or the performance score for the user in association with implementing the current nutritional plan); and (5) Storing all the data in a user profile, and revising the current nutritional plan for the reminder of the time period associated with the nutritional plan based on the past performance data and the degree of deviation from the current nutritional plan.

As set forth earlier, the constant reevaluation of the current nutritional plan and monitoring of the user's opinions and actual implementation of the nutritional plan help the digital assistant to provide better action recommendations and more realistic goals that better fit the user's preferences, motivation, and constraints from other aspects of the user's life. The action recommendations are provided in response to trigger events and under circumstances that promote compliance by the user. For example, the recommendations are provided when the user is interested in hearing food related recommendations and has opportunity, time, and ingredients to comply with the recommendations.

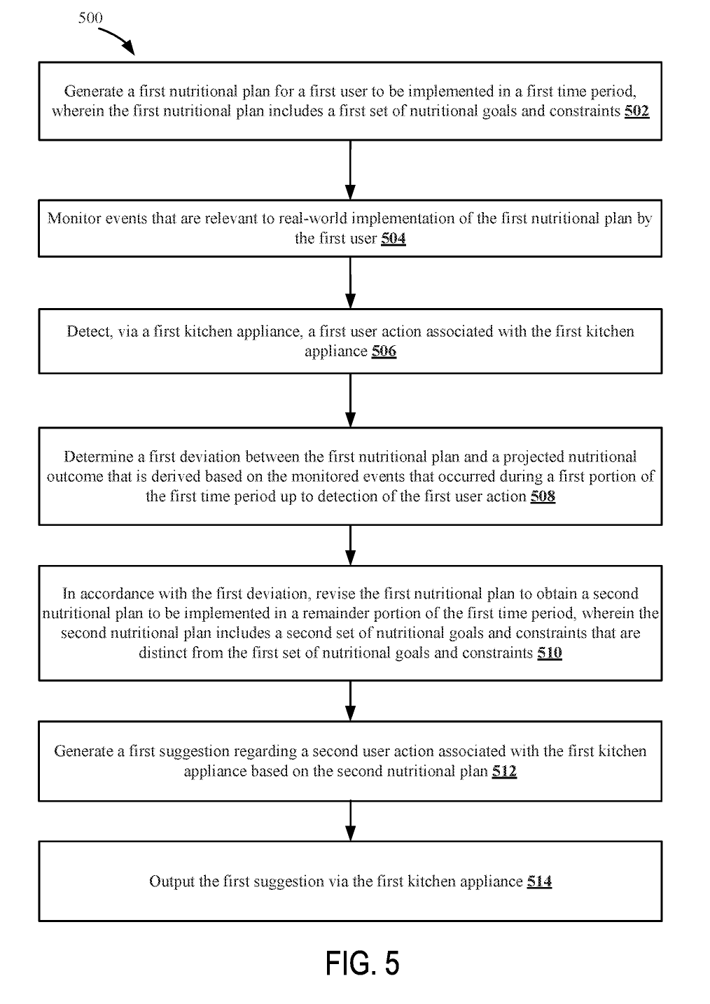

FIG. 5 is a flowchart of a method 500 for providing action recommendations (e.g., restocking recommendations, recipe recommendations, portion recommendations, ingredient recommendations, etc.) in association with kitchen appliances in accordance with some embodiments. The method is performed at a computing system, (e.g., server system 108, FIG. 1) that is communicatively coupled to one or more kitchen appliances (e.g., kitchen appliances 124, FIG. 1).

In some embodiments, the method 500 includes: generating (502) a first nutritional plan (e.g., a first diet plan, a first nutritional composition of various nutrients) for a first user (e.g., a first member of a multi-member household) to be implemented in a first time period (e.g., one month), wherein the first nutritional plan includes a first set of nutritional goals and constraints (e.g., a weight loss goal, a BMI goal, a blood sugar adjustment goal, a limit on sugar intake, a limit on vitamin A intake, etc.).

In some embodiments, the first nutritional plan is generated based on health data of the user and standard nutritional guidance for an average person with similar health condition as the user. In some embodiments, the first set of nutritional goals and constraints include calorie constraints, nutrients arrangements, etc. for a forthcoming period of time, such as a forthcoming month, a forthcoming week, or the next day. In some embodiments, the health data of the user includes a user's height, weight, age, sex, and/or other types of health related data. The health data may further include the user's medical data, such as heart rates, blood pressures, glucose levels, allergies, etc. In some embodiments, the user's health data, such as height, weight, age, and/or sex are entered by the user at the time when the user account was created and registered with the server system 108. In some embodiments, the user's medical data are received from the user via manual input or voice input. In some embodiments, the user's medical data are obtained from the user's electronic medical record, such as via a health related application that is running on the user's mobile device. In some embodiments, the user's health data is received from a wearable device (e.g., a smart watch, a smart pedometer, etc.) associated with the user.

In some embodiments, the standard nutritional guidance for an average person with similar health condition as the user includes how many calories the average person takes per day, what types of nutrients the average person needs every day, and/or how to schedule nutritional meals for a day, etc.

In some embodiments, the first nutrition plan is further generated based on the user's preference data. In some embodiments, the preference data includes the user's preferred cuisine styles (e.g., Chinese, Italian, Japanese, etc.), preferred food categories (e.g., seafood, veggies, meat, diary, etc.), and/or preferred food flavor (e.g., hot, sweet, sour, etc.). In some embodiments, the preference data is obtained via user's voice and/or manual input, user's frequently visited restaurants, and/or data related to user's favorite foods identified from the user's comments on social networking platforms.

In some embodiments, after generating the first nutritional plan, the server system further generates a first set of diet menus in accordance with the first nutritional plan and inventory data associated with the one or more kitchen appliances. In some embodiments, the inventory data are obtained via one or more image sensors located within the user's kitchen appliances, such as the refrigerator, cabinets, ovens, and/or near countertops in the kitchen area (e.g., smart kitchen environment 122) with a field of view that covers the food storage or preparation area. In some embodiments, the inventory data includes information related to meat, vegetables, fruits, rice, bread, seasoning, etc. that can be detected in the kitchen area. In some embodiments, the inventory data includes food category information (e.g., tags regarding whether a certain item is a fruit or vegetable), quantity information (e.g., how many items are left or used). In some embodiments, the inventory data is obtained via one or more other types of sensors located in the kitchen area, such as a weight scale, to provide weight information of the food items. In some embodiments, the first set of diet menus includes a first set of recipes associated with a plurality of meals respectively scheduled for the forthcoming time period. For example, if the first nutritional plan for the user includes recommendations to take more vegetables and to control the calories within 2000 calories per day, and the server system knows that the user's fridge and the cabinets contain mixed greens, tofu, olive oil, and vinegars, the server system may recommend the user to have salad with oil and vinegars for the next lunch.

In one example, at the beginning, a male adult user may create a nutritional goal using a voice input (e.g., “I want to lose 5 lbs. in the next two weeks.”) Then, based on the user's health data and the standard nutrition guidance for an average male adult, the server system generates a nutritional plan including how many calories this male adult user can take per day and what types of nutrients he needs every day. The server system may further generate recommendations of dishes and recipes for three meals every day for the next two weeks.

The method 500 further includes: monitoring (504) events that are relevant to real-world implementation of the first nutritional plan by the first user. In some embodiments, after generating the first nutritional plan, the digital assistant system constantly monitors and collects data from the one or more kitchen appliances (e.g., what food have been taken and cooked, what groceries have been refilled to the fridge and cabinet storage area). The digital assistant system also monitors the user's health data, activity data, and diet data. In some embodiments, the user's health data including the user's recent weight and BMI changes, and the user's recent medical record. The user's activity data includes user's exercise data with how many calories have been consumed. The activity data can be tracked and retrieved from an exercise tracking application that runs on the user's mobile device or wearable devices. In some embodiments, the diet data includes calories and food composition/nutrition data of the meals that have been consumed by the user. In some embodiments, the diet data are tracked by the sensors associated with the kitchen appliances, such as groceries that have been taken out from the fridge and/or the storage cabinets and cooked using the stove and/or oven. The diet data can also be obtained via the user's voice input (e.g., “I/Mom had a mixed green salad, orange chicken, and rice for lunch.”) during a conversation between the digital assistant and the user, or between the digital assistant and another user in the same household.

In some embodiments, the digital assistant also monitors how well the user has adhered to the recommended diet plans, ingredient selections, recipes, portions, and/or cooking methods etc. The digital assistant keeps track of which ones of the recommendations were acted on by the user and which ones of the recommendations were rejected by the user. In some embodiments, the digital assistant initiates dialogues with the user or other members of the household to inquire about the reasons that the user accepted and/or rejected a particular recommended diet plans, ingredient selections, recipes, portions, and/or cooking methods etc.

The method 500 further includes: detecting (506), via a first kitchen appliance (e.g., a smart refrigerator), a first user action (e.g., standing in front of the refrigerator, passing by the refrigerator, opening the refrigerator, taking items out of the refrigerator, putting items into the refrigerator, etc.) associated with the first kitchen appliance. For example, the digital assistant server detects, via a network enabled kitchen appliance in the user's kitchen, that, after a first portion of the first time period (e.g., in the middle of the first week after the implementation of the first nutritional plan was started, or after one week of implementing the first nutritional plan), a first user action associated with a first kitchen appliance. In some embodiments, the first kitchen appliance is related to food storage or meal preparation, such as a stove, a microwave, a fridge, an oven, a toaster oven, or a freezer, etc. In some embodiments, the first user action may be detected by a sensor located near the first kitchen appliance. For example, an image sensor, a light sensor, or a motion sensor detects that the user just opened the refrigerator, or walked close to a storage cabinet.

The method 500 further includes: determining (508) a first deviation between the first nutritional plan and a projected nutritional outcome that is derived based on the monitored events that occurred during a first portion of the first time period up to detection of the first user action. For example, during the first half of the first week from the start of the implementation of the first nutritional plan, the digital assistant determines how well the user has followed the recommendations for implementing the first nutritional plan, and how well the user has independently acted in a manner that is consistent with the first nutritional plan. Based on the actual events and actions that occurred during the first half of the first week from the start of the implementation of the first nutritional plan and the actual effect of the implementation, the digital assistant projects the nutritional outcome that would be achieved if the user continues with the current behavior. The digital assistant determines the amount and/or aspects of deviation(s) between the first nutritional plan and the projected nutritional outcome that is derived based on the earlier implementation of the first nutritional plan.

In some embodiments, the digital assistant continuously monitors the performance of the user with respect to the first nutritional plan. In some embodiments, the digital assistant evaluates the performance of the user with respect to the first nutritional plan in response to predefined trigger events or criteria. For example, the digital assistant optionally performs the evaluation when the digital assistant detects the first user action associated with the first kitchen appliance. In some embodiments, evaluating the first performance includes generating a summary of the past performance during the first portion of the first time period. The summary may include a total calorie intake based on the monitored data, a record of a variety of ingredients (e.g., lettuce, tomatoes, salmon, chicken, etc.) and nutrients (e.g., protein, sugar, carbohydrates, etc.) that have been consumed by the user during the first portion of the first time period. In some embodiments, the digital assistant system further evaluates whether the calories consumed via normal daily activities and exercises are greater or less than the intake of calories. In some embodiments, the digital assistant system calculates an intake of calories for the first portion of the first time period based on the ingredients that have been consumed for the meals taken during the first portion of the first time period. The digital assistant system then compares the calculated intake of calories with a targeted intake of calories associated with the first nutrition plan for the first portion of the first time period to obtain a difference between the two. The digital assistant system also compares the intake of calories with the calories expended on various user activities and exercises. For example, if the user is on a diet to reduce weight and/or body fat, the digital assistant system will calculate the difference between the calories expended via various activities and exercises and the intake of calories every day. In some embodiments, when the first nutritional plan includes goals and constraints for nutrient composition (e.g., balance of fiber, protein, carbohydrates, vitamins, minerals, fats, etc.) for each meal, the digital assistant system evaluates and determines whether the types of nutrients and/or ingredients that have been taken by the user in the past meals meet those goals and constraints.

In some embodiments, evaluations of the performance of the first nutritional plan is scheduled to be conducted periodically, e.g., at 4:30 pm every day before starting preparing dinner, such that a most up-to-date recommendation can be provided to the user for preparing dinner. In some embodiments, after periodically evaluating the performance of the first nutritional plan, a warning is generated and sent to the user when the performance is detected to be below a predetermined threshold. For example, when the digital assistant system detects that the calories consumed for breakfast and lunch have exceeded the target calories for these two meals in the first nutritional plan by over 500 calories, the digital assistant system generates a warning and sends a text message or an audio output to the user when the user enters into the kitchen area to start preparing for dinner. In some embodiments, the evaluation of the performance of the first nutritional plan is triggered by a user's voice input, e.g., the user may ask “what should I have for dinner tonight?”

The method 500 further includes: in accordance with the first deviation, revising (510) the first nutritional plan to obtain a second nutritional plan to be implemented in a remainder portion of the first time period, wherein the second nutritional plan includes a second set of nutritional goals and constraints that are distinct from the first set of nutritional goals and constraints. The overall goals for the first nutritional plan are optionally adjusted based on the reduced duration, and the time average of at least one of the goals and constraints are adjusted to make it easier to implement for the user based on the user's past actions. Alternatively, the overall goals for the second nutritional plan is set for the same duration (e.g., one month) that extends from the current time. In some embodiments, the digital assistant updates, based on the first performance by the user regarding the first nutritional plan during the first portion of the first time period, the first nutritional plan to obtain the second nutritional plan for a second portion of the first time period, wherein the second nutritional plan includes a second set of nutritional goals and constraints. In some embodiments, the second nutritional plan includes calories constraints and nutrient composition constraints that have been modified from the first nutritional plan based on the monitored data. In some embodiments, the second nutritional plan further includes recipes associated with a plurality of meals respectively scheduled for the forthcoming second portion of the first time period. In some embodiments, if the first performance shows that the user consumed too many calories in the first portion of the time period, the second nutritional plan may loosen the overall calorie restriction a little bit (e.g., such that it is easier for the user to implement due to the user's big appetite), while changing the food composition to include more proteins and/or more fiber in the diet (e.g., making the food more filling, and making the user less hungry between meals), as opposed to including too much sugar and simple carbohydrates. In some embodiments, these adjustment strategies for various issues of following a nutritional plan are stored in the substitution database, and the digital assistant can intelligently generate the revised plan based on these strategies and the actual issues faced by the user. If the first performance shows that the user had an unbalanced diet, e.g., too much protein but not enough fiber, the second nutritional plan loosens the limitations on protein intake slightly, but promotes recommendations for ingredients that pair well with proteins and adjusts the relative portions of fiber-containing ingredients to proteins in recommended meal recipes.

The method 500 includes: generating (512) a first suggestion regarding a second user action associated with the first kitchen appliance based on the second nutritional plan, and outputting (514) the first suggestion via the first kitchen appliance. For example, after the digital assistant system detects that the user has opened the fridge, the digital assistant system evaluates the user's performance of the first nutritional plan. If the digital assistant system decides that the user has consumed less fiber than planned during the first portion of the first time period, and the trend and behavior, if continued, would result in a failure in the implementation of the first nutritional plan at the end of the first time period, the digital assistant system revises the first nutritional plan in one or more aspects, such that it will be more likely that it would be successfully implemented to improve the user's health in the same or a similar way (e.g., slightly less effective, or a little more effective) that the first nutritional plan would if properly implemented. The first suggestion that is generated based on the second nutritional plan would be more likely to be followed by the user and still benefit the user's health. For example, the digital assistant may provide a suggestion via an audio output device: “Take green vegetables from the greens compartment.” or “How about a salad and a soup for lunch? A mixed green salad with a vinaigrette dressing, paired with clam chowder, Or a cob salad with chicken strips, paired with tomato soup?”

In some embodiments, the method further includes: detecting, via a second kitchen appliance (e.g., a smart food storage cabinet, a stove, etc.) that is distinct from the first kitchen appliance (e.g., a refrigerator), a third user action associated with the second kitchen appliance (e.g., opening the smart food storage cabinet, or standing in front of the stove); generating a second suggestion regarding an anticipated action of the user based on the third user action and the second nutritional plan (e.g., the suggestions takes into account the previous failures of the user's implementation of the first nutritional plan, and helps the user to do better with respect to the second nutritional plan); and providing the second suggestion to the first user via the second kitchen appliance. For example, after providing the first suggestion to the user, the digital assistant detects that the user has moved from the fridge to the food storage cabinet. The digital assistant system anticipates that the user is going to take out some food items from the food storage cabinet to complement the food items that he/she took out of the refrigerator just now. The digital assistant system thus determines the second action suggestion associated with the second kitchen appliance based on the actual user action of the user at the first kitchen appliance (e.g., the items that the user actually took out of the fridge, and the aspects of the first suggestion that the user has accepted and/or the aspects of the first suggestion that the user has rejected) and based on the second nutritional plan. For example, the digital assistant system detects that the user took vegetables from the fridge, but not the chicken strips, and determines that the user is likely going to take the suggestion to make a green salad with clam chowder, as opposed to a cob salad with tomato soup. Based on the second nutritional plan and the user' action with respect to the first suggestion, the digital assistant system provides a second suggestion for an anticipated action of the user associated with the second kitchen appliance and provides the suggestion via the second kitchen appliance. For example, the digital assistant system detects the user's motion near the seasoning storage area, and then sends an audio output to the user: “Take the olive oil and vinegar to make the vinaigrette” and “Take the canned clam chowder from the pantry.”

In another example, the digital assistant system may have recommended milk and cereal for breakfast in accordance with the first nutritional plan in the first portion of the first time period, but the user has consistently cooked fried eggs for breakfast during the first portion of the first time period. When the digital assistant system detects that the user took two eggs from the fridge, the digital assistant system determines that the user has consumed several heavy meals with eggs in the past two days and that the user is going to fail to meet the calorie and cholesterol goals of the first nutritional plan if the current behavior trend continues. The digital assistant adjusts the first nutritional plan to set looser and more realistic calorie and cholesterol goals for the user. The digital assistant also adjusts the recommendation strategy for the revised nutritional plan. For example, the strategy can reduce recommendations for cereal and milk, but increase recommendations for richer food with less cholesterols, or leaner food with eggs. When the digital assistant detects that the user has moved from the fridge to the stove, the digital assistant generates a second suggestion: “How about a boiled egg? It is better than a fried egg.”

In some embodiments, the digital assistant determines the first deviation between the first nutritional plan and the projected nutritional outcome by: determining a respective compliance level for each of the plurality of goals and constraints based on the monitored events that occurred during the first portion of the first time period; identifying a first constrain in the first nutritional plan, wherein a respective compliance level of the first constraint is below a predefined compliance threshold during the first portion of the first time period; and setting the first constraint in the second nutritional plan based on the respective compliance level of the first constraint during the first portion of the first time period. For example, if the digital assistant determines that the user has consistently failed to follow recommendations that are directed to lower total calorie intake and lower sugar and cholesterol intake by the user, and the compliance levels for all three goals (e.g., calorie intake goal, sugar intake goal, cholesterol intake goal) during the first portion of the first time period are below respective threshold levels, the digital assistant resets the three goals, by adjusting the goals up by 10%. Alternatively, the digital assistant determines that the user has followed at least some of the recommendations with lower sugar and low cholesterol ingredients and recipes during the first portion of the first period of time. Based on such a determination, the digital assistant selects a recommendation strategy that utilizes more low sugar and low cholesterol options with higher fat content or calories, such that it is more likely that the user would accept those recommendations, and still achieve an overall reduction in calorie intake as compared to the scenario where the user continues to ignore the recommendations generated based on the first nutritional plan and corresponding recommendation strategies.

In some embodiments, the digital assistant system evaluates the performance by the user related to the first nutritional plan by determining a constraint factor, such as a difference in calories between the actual intake by the user and the target calories from the nutritional plan for the same period of time. The digital assistant system then updates the first nutritional plan to obtain the second nutritional plan by adjusting, e.g., increasing or decreasing, the constraint factor associated with the second nutritional plan. For example, if during the past two days, the user always took about 100 calories more for dinner than what was recommended in the first nutritional plan, for the dinners in the next two days, the digital assistant system recommends a dinner with 80 calories more than the dinner in the first nutritional plan, so that the it becomes easier for the user to form a habit of sticking to the nutritional plan. In some embodiments, the digital assistant system also gives incentives (e.g., recommending a desert after dinner) when the user followed the nutritional plan during the past two meals.

In some embodiments, the first kitchen appliance is a food storage appliance, and the digital assistant generates the first suggestion regarding the second user action associated with the first kitchen appliance based on the second nutritional plan by: determining inventory of items currently stored in the first kitchen appliance; and generating a restocking suggestion regarding one or more items to be refilled in the first kitchen appliance in accordance with the inventory of items currently stored in the first kitchen appliance and the second nutritional plan. For example, the second nutritional plan may require a strategy to increase fiber content of the ingredients and to pose less restriction on calories, as compared to the strategy that is purely based on calorie restrictions for the first nutritional plan. The digital assistant system checks the inventory in the fridge to determine whether a refill of ingredients with more fiber content (e.g., sweet potatoes, avocados, whole grain breads, as opposed to only lettuces and cucumbers) is needed. In some embodiments, the first suggestion regarding the second user action associated with the first kitchen appliance comprises audio notification to the user to refill the first kitchen appliance (e.g., the fridge) with one or more food ingredients (e.g., the ingredients identified from the new strategy and the current inventory data). In some embodiments, the digital assistant system creates a shopping list for the user and sends it to the user's mobile device. In some embodiments, the digital assistant asks for the user's confirmation for online purchase through the digital assistant.

In some embodiments, the digital assistant system updates the first nutritional plan based on user's feedbacks regarding the first nutritional plan. For example, the digital assistant system may solicit comments from the user after the user follows the nutritional plan for one day, such as using an audio output: “How did you like your meals today?” After receiving the user's feedbacks (e.g., “I like something spicier”), the first nutritional plan and corresponding recommendation strategies may be updated, and the second nutritional plan may include more spicy dishes (e.g., Gong Pao chicken to replace the orange chicken in the first nutritional plan).

In some embodiments, the digital assistant sets up a group account including a plurality of users within a household including at least the first user and a second user, and the digital assistant generates the second nutritional plan based on data on each of the plurality of users within the household. For example, in many households, the meal preparation impacts multiple members of the household because everyone eats at the same table. In some embodiments, the individual health needs are taken into account when the digital assistant recommends strategies for prepare meals for the whole household. In many cases, the preferences and constraints of multiple members are taken into consideration by the digital assistant when the digital assistant recommends the ingredients, recipes, cooking methods, and portions, to one of the household members that does the meal preparation. In some embodiments, the digital assistant provides recommendations regarding the respective portions each member of the household should take from the prepared meal. In some embodiments, the digital assistant outputs the portion recommendations to the person that is setting the table. In some embodiments, the digital assistant outputs the portion recommendation for each household member when they have all sat down at the table.

In some embodiments, the digital assistant requests user feedback regarding the first nutritional plan and real-world implementation of the first nutritional plan by the first user, including requesting, via a chatbot, user feedback from at least the second user within the household regarding the real-world implementation of the first nutritional plan by the first user. For example, if first member of the household often prepares the meal in the kitchen, and the meal is consumed by both the first member and a second member of the household, the digital assistant optionally converses with the first member and inquire about the first member's opinions and actions regarding the food recommendations in the past. Similarly, if the second member often does the dishes after the meal, the digital assistant also converses with the second member and inquire about the second member's opinions and actions regarding the food recommendations in the past.

In some embodiments, the first user action associated with the first kitchen appliance is performed by the second user that is distinct from the first user, and the first suggestion regarding the second user action is to be performed by the second user. For example, the first user is the household member that consumes meals prepared by the second user, and the second user receives the recommendations based on the second nutritional plan for the first user, and prepares the meal for the first user.

In some embodiments, upon receiving a user voice command regarding generating a dinner plan for the household, the digital assistant system analyzes the nutritional plans and respective monitored data for all users in the household. In some embodiments, the digital assistant system manages the user's data for each user account within the household based on biometric data (e.g., audio fingerprints, face recognition, etc.) of respective users.

In some embodiments, the digital assistant system may consult another user in the household when generating recommendations for the user. For example, the digital assistant system may detects the first user opened the fridge and stared at the inside of the fridge for 20 seconds, seemingly uncertain about what ingredients to take from the fridge to prepare dinner. The digital assistant system may automatically generate a notification to another user (e.g., a text message or an audio output) to ask for ideas for dinner. The other user may provide a recommendation of grilled salmon for dinner to the digital assistant system via text message or voice input. The digital assistant system then generates a suggestion (e.g., “How about grilled salmon for tonight?”) to output to the first user based on the second user's input.

Other details of the method and the food preparation system are described in other parts of the disclosure and is not repeated here in the interest of brevity. It should be understood that the particular order in which the operations in FIG. 5 have been described is merely exemplary and is not intended to indicate that the described order is the only order in which the operations could be performed. One of ordinary skill in the art would recognize various ways to reorder the operations described herein. Additionally, it should be noted that details of other processes described herein with respect to other methods and/or processes described herein are also applicable in an analogous manner to method 500 described above.

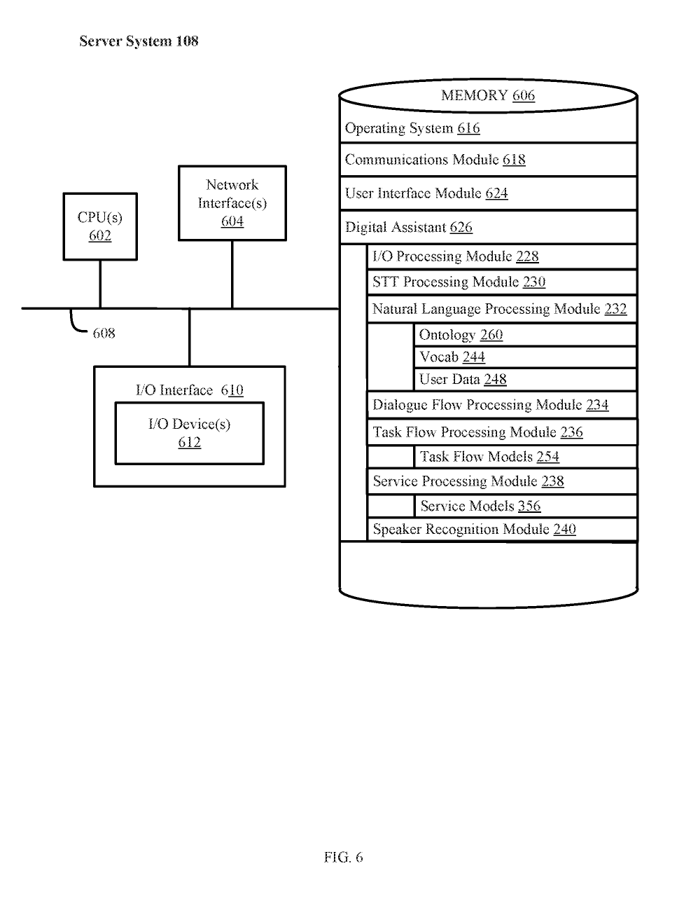

FIG. 6 is a block diagram of the server system 108 for providing action recommendations associated with kitchen appliances in accordance with some embodiments. The server system 108 includes one or more processing units (CPUs) 602, one or more network interfaces 604, memory 606, one or more input/output (I/O) interfaces 610, and one or more communication buses 608 for interconnecting these components (sometimes called a chipset).

In some embodiments, the network communications interface 604 includes wired communication port(s) and/or wireless transmission and reception circuitry. The wired communication port(s) receive and send communication signals via one or more wired interfaces, e.g., Ethernet, Universal Serial Bus (USB), FIREWIRE, etc. The wireless circuitry typically receives and sends RF signals and/or optical signals from/to communications networks and other communications devices. The wireless communications may use any of a plurality of communications standards, protocols and technologies, such as GSM, EDGE, CDMA, TDMA, Bluetooth, Wi-Fi, VoIP, Wi-MAX, or any other suitable communication protocol. The network communications interface 404 enables communication between the server system 108 with networks, such as the Internet, an intranet and/or a wireless network, such as a cellular telephone network, a wireless local area network (LAN) and/or a metropolitan area network (MAN), and other devices.

In some embodiments, the I/O interface 610 couples input/output devices 612, such as displays, a keyboards, touch screens, speakers, and microphones, to the user interface module 624. The I/O interface 610, in conjunction with the user interface module 624, receives user inputs (e.g., voice input, keyboard inputs, touch inputs, etc.) from the kitchen appliances and/or user mobile devices, and process them accordingly.

In some embodiments, memory 606 includes non-volatile memory, such as one or more magnetic disk storage devices, one or more optical disk storage devices, one or more flash memory devices, or one or more other non-volatile solid state storage devices. Memory 406, optionally, includes one or more storage devices remotely located from one or more processing units 602. Memory 606, or alternatively the non-volatile memory within memory 606, includes a non-transitory computer readable storage medium. In some implementations, memory 606, or the non-transitory computer readable storage medium of memory 606, stores the following programs, modules, and data structures, or a subset or superset thereof:
operating system 616 including procedures for handling various basic system services and for performing hardware dependent tasks;
network communication module 618 for connecting to external services via one or more network interfaces 604 (wired or wireless);
user interface module 624 for enabling presentation of information and receiving inputs;
digital assistant server-side 626 that interface with the digital assistant client-sides on various user devices and home appliances;
I/O processing module 228 for transmitting and receiving event data, speech inputs, context data, follow-up dialogues, action recommendations, etc.;
speaker recognition module 240 for recognizing user's identities based on biometric data, voiceprints, etc.;
STT processing module 230 for converting user's speech inputs to text strings;
natural language processing module 232 for identifying user's intent and information items disclosed in the user's voice inputs;
task flow processing modules 236 for generating instructions for performing a task in accordance with user's intent;
dialogue processing module 234 for generating dialogue and speech outputs for elicit additional information, clarification of existing inputs, and requesting user feedback re current nutritional plan and recommendations, etc.;
service processing module 238 for utilizing external services to perform a task in accordance with user's intent and/or the digital assistant's recommendation; and
action recommendation module 242 for monitoring events, user data, and nutritional plans, and revising nutritional plans and recommendation strategies, and providing action recommendations for implementing the nutritional plans.

Each of the above identified elements may be stored in one or more of the previously mentioned memory devices, and corresponds to a set of instructions for performing a function described above. The above identified modules or programs (i.e., sets of instructions) need not be implemented as separate software programs, procedures, modules or data structures, and thus various subsets of these modules may be combined or otherwise re-arranged in various implementations. In some implementations, memory 606, optionally, stores a subset of the modules and data structures identified above. Furthermore, memory 606, optionally, stores additional modules and data structures not described above.

The foregoing description, for purpose of explanation, has been described with reference to specific embodiments. However, the illustrative discussions above are not intended to be exhaustive or to limit the disclosed embodiments to the precise forms disclosed. Many modifications and variations are possible in view of the above teachings. The embodiments were chosen and described in order to best explain the principles and practical applications of the disclosed ideas, to thereby enable others skilled in the art to best utilize them with various modifications as are suited to the particular use contemplated.

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “upon a determination that” or “in response to determining” or “in accordance with a determination” or “upon detecting” or “in response to detecting” that the stated condition precedent is true, depending on the context.

What is claimed is:

1. A method, comprising:
at a computing device comprising one or more processors and memory:
generating a first nutritional plan for a first user to be implemented in a first time period, wherein the first nutritional plan includes a first set of nutritional goals and constraints;
monitoring events that are relevant to real-world implementation of the first nutritional plan by the first user;
detecting, via a first kitchen appliance, a first user action associated with the first kitchen appliance;
determining a first deviation between the first nutritional plan and a projected nutritional outcome that is derived based on the monitored events that occurred during a first portion of the first time period up to detection of the first user action;
in accordance with the first deviation, revising the first nutritional plan to obtain a second nutritional plan to be implemented in a remainder portion of the first time period, wherein the second nutritional plan includes a second set of nutritional goals and constraints that are distinct from the first set of nutritional goals and constraints;
generating a first suggestion regarding a second user action associated with the first kitchen appliance based on the second nutritional plan; and
outputting the first suggestion via the first kitchen appliance.

2. The method of claim 1, including:
detecting, via a second kitchen appliance that is distinct from the first kitchen appliance, a third user action associated with the second kitchen appliance; and
generating a second suggestion regarding an anticipated action of the user based on the third user action and the second nutritional plan; and
providing the second suggestion to the first user via the second kitchen appliance.

3. The method of claim 1, wherein determining the first deviation between the first nutritional plan and the projected nutritional outcome that is derived based on the monitored events that occurred during the first portion of the first time period up to detection of the first user action includes:
determining a respective compliance level for each of the plurality of goals and constraints based on the monitored events that occurred during the first portion of the first time period;
identifying a first constrain in the first nutritional plan, wherein a respective compliance level of the first constraint is below a predefined compliance threshold during the first portion of the first time period; and
setting the first constraint in the second nutritional plan based on the respective compliance level of the first constraint during the first portion of the first time period.

4. The method of claim 1, wherein the first kitchen appliance is a food storage appliance, and wherein generating the first suggestion regarding the second user action associated with the first kitchen appliance based on the second nutritional plan includes:
determining inventory of items currently stored in the first kitchen appliance; and
generating a restocking suggestion regarding one or more items to be refilled in the first kitchen appliance in accordance with the inventory of items currently stored in the first kitchen appliance and the second nutritional plan.

5. The method of claim 1, including:
setting up a group account including a plurality of users within a household including at least the first user and a second user; and
generating the second nutritional plan based on data on each of the plurality of users within the household.

6. The method of claim 5, including:
requesting user feedback regarding the first nutritional plan and real-world implementation of the first nutritional plan by the first user, including requesting, via a chatbot, user feedback from at least the second user within the household regarding the real-world implementation of the first nutritional plan by the first user.

7. The method of claim 5, wherein the first user action associated with the first kitchen appliance is performed by the second user that is distinct from the first user, and the first suggestion regarding the second user action is to be performed by the second user.

8. A system, comprising:
one or more processors; and
memory storing instructions, the instructions, when executed by the one or more processors cause the processors to perform operations comprising:
generating a first nutritional plan for a first user to be implemented in a first time period, wherein the first nutritional plan includes a first set of nutritional goals and constraints;
monitoring events that are relevant to real-world implementation of the first nutritional plan by the first user;
detecting, via a first kitchen appliance, a first user action associated with the first kitchen appliance;
determining a first deviation between the first nutritional plan and a projected nutritional outcome that is derived based on the monitored events that occurred during a first portion of the first time period up to detection of the first user action;
in accordance with the first deviation, revising the first nutritional plan to obtain a second nutritional plan to be implemented in a remainder portion of the first time period, wherein the second nutritional plan includes a second set of nutritional goals and constraints that are distinct from the first set of nutritional goals and constraints;
generating a first suggestion regarding a second user action associated with the first kitchen appliance based on the second nutritional plan; and
outputting the first suggestion via the first kitchen appliance.

9. The system of claim 8, wherein the operations include:
detecting, via a second kitchen appliance that is distinct from the first kitchen appliance, a third user action associated with the second kitchen appliance; and
generating a second suggestion regarding an anticipated action of the user based on the third user action and the second nutritional plan; and
providing the second suggestion to the first user via the second kitchen appliance.

10. The system of claim 8, wherein determining the first deviation between the first nutritional plan and the projected nutritional outcome that is derived based on the monitored events that occurred during the first portion of the first time period up to detection of the first user action includes:
determining a respective compliance level for each of the plurality of goals and constraints based on the monitored events that occurred during the first portion of the first time period;
identifying a first constrain in the first nutritional plan, wherein a respective compliance level of the first constraint is below a predefined compliance threshold during the first portion of the first time period; and
setting the first constraint in the second nutritional plan based on the respective compliance level of the first constraint during the first portion of the first time period.

11. The system of claim 8, wherein the first kitchen appliance is a food storage appliance, and wherein generating the first suggestion regarding the second user action associated with the first kitchen appliance based on the second nutritional plan includes:
determining inventory of items currently stored in the first kitchen appliance; and
generating a restocking suggestion regarding one or more items to be refilled in the first kitchen appliance in accordance with the inventory of items currently stored in the first kitchen appliance and the second nutritional plan.

12. The system of claim 8, wherein the operations include:
setting up a group account including a plurality of users within a household including at least the first user and a second user; and
generating the second nutritional plan based on data on each of the plurality of users within the household.

13. The system of claim 12, wherein the operations include:
requesting user feedback regarding the first nutritional plan and real-world implementation of the first nutritional plan by the first user, including requesting, via a chatbot, user feedback from at least the second user within the household regarding the real-world implementation of the first nutritional plan by the first user.

14. The system of claim 12, wherein the first user action associated with the first kitchen appliance is performed by the second user that is distinct from the first user, and the first suggestion regarding the second user action is to be performed by the second user.

15. A non-transitory computer-readable storage medium storing instructions, the instructions, when executed by one or more processors of a computing system, cause the computing system to perform operations comprising:
generating a first nutritional plan for a first user to be implemented in a first time period, wherein the first nutritional plan includes a first set of nutritional goals and constraints;
monitoring events that are relevant to real-world implementation of the first nutritional plan by the first user;
detecting, via a first kitchen appliance, a first user action associated with the first kitchen appliance;
determining a first deviation between the first nutritional plan and a projected nutritional outcome that is derived based on the monitored events that occurred during a first portion of the first time period up to detection of the first user action;
in accordance with the first deviation, revising the first nutritional plan to obtain a second nutritional plan to be implemented in a remainder portion of the first time period, wherein the second nutritional plan includes a second set of nutritional goals and constraints that are distinct from the first set of nutritional goals and constraints;
generating a first suggestion regarding a second user action associated with the first kitchen appliance based on the second nutritional plan; and
outputting the first suggestion via the first kitchen appliance.

16. The computer-readable storage medium of claim 15, wherein the operations include:
detecting, via a second kitchen appliance that is distinct from the first kitchen appliance, a third user action associated with the second kitchen appliance; and
generating a second suggestion regarding an anticipated action of the user based on the third user action and the second nutritional plan; and
providing the second suggestion to the first user via the second kitchen appliance.

17. The computer-readable storage medium of claim 15, wherein determining the first deviation between the first nutritional plan and the projected nutritional outcome that is derived based on the monitored events that occurred during the first portion of the first time period up to detection of the first user action includes:
determining a respective compliance level for each of the plurality of goals and constraints based on the monitored events that occurred during the first portion of the first time period;
identifying a first constrain in the first nutritional plan, wherein a respective compliance level of the first constraint is below a predefined compliance threshold during the first portion of the first time period; and
setting the first constraint in the second nutritional plan based on the respective compliance level of the first constraint during the first portion of the first time period.

18. The computer-readable storage medium of claim 15, wherein the first kitchen appliance is a food storage appliance, and wherein generating the first suggestion regarding the second user action associated with the first kitchen appliance based on the second nutritional plan includes:
determining inventory of items currently stored in the first kitchen appliance; and
generating a restocking suggestion regarding one or more items to be refilled in the first kitchen appliance in accordance with the inventory of items currently stored in the first kitchen appliance and the second nutritional plan.

19. The computer-readable storage medium of claim 15, wherein the operations include:
setting up a group account including a plurality of users within a household including at least the first user and a second user; and
generating the second nutritional plan based on data on each of the plurality of users within the household.

20. The computer-readable storage medium of claim 19, wherein the operations include:
requesting user feedback regarding the first nutritional plan and real-world implementation of the first nutritional plan by the first user, including requesting, via a chatbot, user feedback from at least the second user within the household regarding the real-world implementation of the first nutritional plan by the first user.
